(19) United States
(12) Patent Application Publication
(10) Pub. No.: US 2026/0315741 A1
(43) Pub. Date: Oct. 15, 2026

(54) AXIALLY MOVABLE AND PRESSURE-TIGHT CONNECTION OF VI CATHETER SHAFTS

(71) Applicant: BIOTRONIK AG, Buelach (CH)

(72) Inventors: Jeremy Wernli, Wettingen (CH); Tisha Kalongi, Zuerich (CH); Matthias Wesselmann, Ruedlingen (CH)

(21) Appl. No.: 19/104,495

(22) PCT Filed: Aug. 9, 2023
(86) PCT No.: PCT/EP2023/072067

§ 371 Date: Feb. 18, 2025

(30) Foreign Application Priority Data
Aug. 24, 2022 (EP) 22191901.2

(51) Int. Cl. A61M 25/01; A61M 25/00

(52) U.S. Cl. CPC A61M 25/0119; A61M 2025/0004; A61M 2210/12

(57) ABSTRACT

A catheter includes an inner shaft and an outer shaft. The inner shaft includes a distal inner shaft portion and a portion arranged proximally to the distal inner shaft portion. The catheter includes a proximal rolling membrane attached to portion arranged proximally to the distal inner shaft portion and to the outer shaft.

### PRIORITY CLAIM

This application is a 35 U.S.C. 371 US National Phase and claims priority under 35 U.S.C. § 119, 35 U.S.C. 365 (b) and all applicable statutes and treaties from prior PCT Application PCT/EP2023/072067, which was filed Aug. 9, 2023, which application claimed priority from EP Application Serial No. 22191901.2, which was filed Aug. 24, 2022.

### FIELD OF THE INVENTION

A field of the invention concerns catheters, including catheters used for vascular interventions (VI).

### BACKGROUND

Conventional catheters are often based on an at least dual-shaft arrangement wherein an inner shaft is arranged within an outer shaft. Such an arrangement may provide the advantage that, e.g., a guide-wire may be arranged in the inner shaft to allow a guiding of the catheter to a preferred location in the body of the patient while being able to, independently from the guide-wire, provide a distal end of the catheter with, e.g., a rinsing fluid (which may be inserted into a lumen defined in between the inner shaft and the outer shaft). However, if a lumen defined in between the inner shaft and the outer shaft is filled with a liquid, it has also to be ensured that a respective proximal end of the catheter is provided with a respective sealing.

Moreover, it is often desired that the at least two shafts are movable relative to each other such that a length of the catheter may be adaptable to the current needs of the vascular intervention to be performed. Moreover, such a movability of the inner shaft relative to the outer shaft may further allow a variation of the location of a distal tip of the outer shaft relative to a distal tip of the inner shaft to, e.g., selectively deliver a medical device (e.g., a pacemaker) and/or a medical drug (which may be located at the distal tip of the inner shaft). In some applications, it is further desirable to provide both functionalities at the very same time: sealing of the lumen at a proximal end of the catheter while still allowing a movability of the inner shaft relative to the outer shaft. For said purpose, a conventional catheter may be provided with a valve system such as, e.g., a haemostatic valve, on the proximal end of the catheter which may allow a proximal sealing of the lumen while allowing a certain (restricted) movability of the inner shaft relative to the outer shaft. In some applications, at least a friction-reduced sealing of the lumen between the inner shaft and the outer shaft of a catheter may be provided by providing the lumen with a swelling material (e.g., a swelling hydrogel). In particular, the latter is rather complicated in its manufacturing and may lead to particle emission.

In modern cardiovascular interventions, it is desired to provide the movability of the inner shaft relative to the outer shaft in a substantially friction-less manner such as to allow a physician an easy control of the movement while still being focused on the actual vascular intervention. At the very same time it is desired that a sealing of the lumen defined in between the inner shaft and the outer shaft is maintained. However, since the liquid tight sealing methods known in the art (e.g., based on haemostatic valves) may at most be a compromise between sealing and friction-less movability (the sealing may sometimes even counteract the desired substantial friction-less movability of the inner shaft relative to the outer shaft) currently used catheters are limited in their applications.

### SUMMARY OF THE INVENTION

A catheter of a preferred embodiment includes an inner shaft and an outer shaft. The inner shaft includes a distal inner shaft portion and a proximal inner shaft portion arranged proximally to the distal inner shaft portion, the catheter includes a proximal rolling membrane attached to the proximal inner shaft portion arranged proximally to the distal inner shaft portion. The proximal rolling membrane enables a sealing of the proximal end of the catheter.

### BRIEF DESCRIPTION OF THE DRAWINGS

The following figures are provided to support the understanding of the present invention:

FIGS. 1A-1B: Illustration of an exemplary embodiment of a catheter with a proximal rolling membrane;

FIGS. 2A-2B: Illustration of a further exemplary embodiment of a catheter with a proximal rolling membrane;

FIGS. 3A-3B: Illustration of a further exemplary embodiment of a catheter with a proximal rolling membrane;

FIGS. 4A-4B: Illustration of a further exemplary embodiment of a catheter with a proximal rolling membrane;

FIGS. 5A-5C: Illustration of a further exemplary embodiment of a catheter with a proximal rolling membrane and a distal rolling membrane.

### DETAILED DESCRIPTION OF THE PREFERRED EMBODIMENTS

A catheter of a preferred embodiment includes or consists of an inner shaft, an outer shaft, and one or both of a proximal rolling membrane and a distal rolling membrane. The inner shaft includes a distal inner shaft portion and a proximal inner shaft portion arranged proximally to the distal inner shaft portion. The proximal rolling membrane is attached to the distal inner shaft portion and to the outer shaft. The proximal rolling membrane enables a sealing of the proximal end of the catheter. The distal rolling membrane is attached to the distal inner shaft portion and to the outer shaft or a second outer shaft.

The catheter can be a catheter which can be inserted into a body of a patient. The catheter can be a catheter which can be inserted into a blood vessel of the patient. In some exemplary implementations, the catheter can be a catheter for a coronary intervention and/or a (cardio-) vascular intervention.

The catheter has a proximal end and an opposing distal end. The proximal end of the catheter (or proximal catheter end) is to be understood as the end of the catheter which is furthest away from the body of the patient and which is closest to the operator of the catheter. The proximal rolling membrane enables a sealing of the proximal end of the catheter.

A distal inner shaft portion can be understood as a portion of the catheter which is arranged closest to a center of the body of the patient. In other words, the distal inner shaft portion of the catheter can be understood as the location of the catheter which is furthest away from an operator (e.g., a physician). In contrast, a proximal portion of the catheter is to be understood as the portion of the catheter which is furthest away from the center of the body of the patient and which is closest to the operator of the catheter. Based thereon, an intermediate portion can be understood as the portion of the catheter which lies in between the distal inner shaft portion of the catheter and the proximal portion of the catheter. Each of the distal inner shaft portion, the proximal portion and the intermediate portion can be adapted to be equal in size/length, e.g., the total length of the inner shaft can be divided into three parts of equal length. Alternatively, also any other ratio of the distal inner shaft portion, the proximal portion and the intermediate portion relative to each other can be possible.

The proximal rolling membrane can be attached to the inner shaft in an intermediate inner shaft portion and/or in a proximal inner shaft portion. The proximal rolling membrane can be attached to the outer shaft in an intermediate outer shaft portion and/or in a proximal outer shaft portion. In a preferred case, irrespective of the portion in which the proximal rolling membrane is attached to the inner shaft, the catheter can be adapted such that the proximal rolling membrane is not attached to the inner shaft at a distal inner shaft portion of the inner shaft. The proximal rolling membrane can generally be attached around an outer circumference of the inner shaft, for example, and around an inner and/or outer circumference of the outer shaft, for example.

Such a catheter can facilitate a termination of a lumen defined in between the inner shaft and the outer shaft towards the proximal end of the catheter, e.g., at which end a Luer (port) can be located. In other words, any fluids (e.g., liquids and/or gases) which can be present in the inner lumen defined between the inner shaft and the outer shaft can be prevented from flowing to the proximal end (e.g., past the proximal rolling membrane) of the catheter at which an operator of the catheter can be situated. Therefore, a clean working environment at the proximal end of the catheter can be ensured, and a leak of body liquids is prevented. Vice versa, it can also be facilitated that a sterile working environment in the inner lumen can be maintained. The sterile working environment can be based on preventing any air and/or liquids (which can each contain harmful bacteria and/or viruses), which can enter the lumen from a hypothetically open proximal end, from arriving at a distal end of the catheter from which they can migrate into the blood vessel of the patient.

It is further noted that the catheter can not be limited to the aspects described above can be combined with aspects from catheters according to other aspects described herein.

Another aspect relates to a catheter which can include an inner shaft and an outer shaft. Moreover, the catheter can include a proximal rolling membrane which can be attached to the inner shaft and the outer shaft, wherein the proximal rolling membrane can be attached to the inner shaft in a portion of the catheter that can be configured to remain outside of a patient.

This can allow a nearly frictionless relative movement of inner and outer shafts.

Additionally or alternatively, the proximal rolling membrane can be attached to the outer shaft in a portion of the catheter that can be configured to remain outside of a patient. This can ensure that the inner shaft can be conveniently moved relative to the outer shaft by an operator in a frictionless manner, while providing a good sealing of the proximal portion of the outer shaft.

In some cases, the inner shaft can be arranged concentrically (e.g., centered) in the outer shaft. The inner shaft can, in some cases, include an inner lumen or can alternatively be implemented as a solid rod-like element.

The inner shaft and/or the outer shaft can be made from the same material as the proximal rolling membrane. Alternatively or additionally, the inner shaft and/or the outer shaft can be made from any kind of solid state material (e.g., plastic materials, rubber-like materials, etc.). In preferred cases, the inner shaft and/or the outer shaft can be made from a material which is pressure-resistant and which can allow for a pressure tight attachment of the proximal rolling membrane thereto. In some cases, the inner shaft and/or the outer shaft can in particular be made (at least in part) from a biocompatible material (e.g., a material which does not involve a danger for the health (e.g., due to allergic reactions, carcinogenic materials, etc.) of the patient even in cases in which it can be in contact with the body of the patient.

The proximal rolling membrane and/or a distal rolling membrane can be made from a thermoplastic material (which can, e.g., also be used for balloons for applications in angioplasty). The proximal rolling membrane can be made from a pressure resistant and/or bendable and/or ductile material which can be a material (or material composition) exemplarily selected from the group of polyamides and/or polyurethanes and/or any other suitable material. In analogy to the inner shaft and/or the outer shaft, the proximal rolling membrane can be made at least in part from a biocompatible material. In other cases, the material for manufacturing the proximal rolling membrane can not be selected to be biocompatible (as the proximal rolling membrane will be located outside the patient during the medical intervention).

By attaching the proximal rolling membrane to a portion of the inner or outer shaft which can remain outside of the body of the patient, the material selection for manufacturing the respective proximal rolling membrane and/or the portion of the inner/outer shaft can be simplified as no specific biocompatible materials and/or material combinations can be required for the proximal rolling membrane and/or the portion of the shafts which remains outside of the body of the patient. This can contribute to decreased manufacturing costs (and thus a reduced product price) and increased recycling capabilities as less materials can be required to be separated and reprocessed (differently).

It is further noted that the aspects of the catheter of these aspects can also be combined with aspects of other embodiments as described herein.

In some cases, the inner shaft and the outer shaft can be adapted to be movable relative to each other along an axial direction.

An axial direction can be understood as a direction along a longitudinal extension (e.g. defined by a direction from the proximal end to the distal end of the catheter) of the catheter (and thus of the inner shaft and the outer shaft). In other words, the inner shaft can be movable along a distal direction and back along a proximal direction.

By allowing a movement of the inner shaft relative to the outer shaft along an axial direction, a positioning of the inner shaft relative to the outer shaft can be facilitated. Said positioning can, e.g., be used to deliver a certain medical device and/or drug to the distal most portion of the catheter by moving the inner shaft in the distal direction relative to the outer shaft. Said movement can preferably be initiated from a proximal end of the catheter and, in some cases, from outside of the patient's body.

Another aspect relates to a catheter which can include an inner shaft and an outer shaft, wherein the inner shaft and the outer shaft can be adapted to be movable relative to each other in an axial direction. Moreover, the catheter can include a proximal rolling membrane which can be attached to the inner shaft and the outer shaft, wherein the catheter can be adapted such that the proximal rolling membrane cannot leave the catheter in a distal direction when the inner and outer shafts are moved relative to each other. Hence, the proximal rolling membrane can specifically be adapted to provide relative movement in relatively proximal regions.

In some cases, the proximal rolling membrane can be adapted such that it can (only) leave the catheter in a proximal direction when the inner shaft and the outer shaft are moved relative to each other.

By adapting the catheter such that the proximal rolling membrane cannot leave the catheter in a distal direction, a catheter can be provided with a proximal rolling membrane which can predominantly be arranged at a proximal portion of the catheter. In some cases, the proximal rolling membrane can reside in a handle and/or a handle area of the catheter.

It is further noted that the aspects of the catheter of this embodiment can also be combined with aspects of other embodiments as described herein.

The catheter can be configured such that the proximal rolling membrane can be located outside of a patient.

In some cases, the proximal rolling membrane can be adapted such that it entirely lies outside of the patient. Alternatively, it can be possible that only a portion of the proximal rolling membrane lies outside of the patient whereas a respective other portion of the proximal rolling membrane lies inside the body of the patient.

By configuring the catheter such that the proximal rolling membrane lies outside of the patient, stringent demands on the material (and/or the material composition) the proximal rolling membrane is made from can be reduced (e.g., no specific biocompatible materials can be required anymore) with reduced manufacturing and product costs for the catheter. Moreover, if the proximal rolling membrane lies outside of the patient, the proximal rolling membrane can be accessible more easily by a physician such that an easier intervention can be possible if a blocking of the movability of the inner shaft relative to the outer shaft occurs which can at least in part be based on the proximal rolling membrane.

The catheter can be configured such that the proximal rolling membrane can include a portion for an attachment to the inner shaft, wherein an end of the portion faces a distal direction.

In some cases, the proximal rolling membrane can be configured such that it can extend (e.g. can be rolled out) longitudinally. The proximal rolling membrane can be provided with a proximal end and with an opposing distal end.

The proximal end of the proximal rolling membrane can be the end of the proximal rolling membrane which includes the portion for attachment of the proximal rolling membrane to the inner shaft. In analogy, the distal end of the proximal rolling membrane can be the end of the proximal rolling membrane which is attached to the outer shaft of the catheter.

In some cases, the catheter can be configured such that the proximal end of the proximal rolling membrane can be folded inwards into itself, such that the inwards folded portion faces the distal direction (e.g., can be aligned towards the center of the body of the patient) and such that an overlapping portion can be created at the inwards folded proximal end of the rolling membrane.

The folding of at least a portion of the proximal rolling membrane inwards into itself and attaching the inwards folded portion of the proximal rolling membrane to the inner shaft can ensure that the inwards folded characteristic of the proximal rolling membrane is maintained as the inner shaft is moved relative to the outer shaft along, e.g., an axial direction. In this context, an undesired compression of the proximal rolling membrane in a distal direction can be avoided (e.g., if the outer shaft is moved in a distal direction) which can otherwise lead to a formation of one or more folded layers of the proximal rolling membrane along the distal direction (similar to an accordion). Said formation can disadvantageously lead to a seizing of the respective layers such that a further movement of the inner shaft relative to the outer shaft can be disadvantageously affected or even inhibited. Such an arrangement can contribute to a convenient and substantially friction-less movability of the inner shaft relative to the outer shaft.

The catheter can be configured such that the proximal rolling membrane can include a portion for attachment to the inner shaft, wherein an end of the portion faces a proximal direction.

In some cases, the proximal end of the proximal rolling membrane can directly face the proximal direction (i.e., an inner side of a hose-like proximal rolling membrane can be attached to an outer side of the inner shaft).

The portion for attachment can be implemented as a (spatially limited) attachment point to the very proximal end of the inner shaft. Alternatively, the portion for attachment can be implemented as an attachment area (e.g., the proximal rolling membrane can be attached to the inner shaft at an area with a surface of approximately 1 cm², 2 cm², 3 cm² or any other suitable value). Preferably, at least a portion of the proximal end of the proximal rolling membrane can circumvent the inner shaft such that a ring-like portion for attachment is formed in which the proximal rolling membrane can be attached to the inner shaft. Said ring-like portion can be provided in a spatially limited manner (e.g., with no substantial longitudinal extension) and thus an essentially circular portion for attachment or the ring-like element can be provided with a longitudinal extension (e.g., can be configured as a cylinder jacket-like portion for attachment).

Such an arrangement can contribute to a friction-less movability of the inner shaft relative to the outer shaft and can contribute to a sealing of the catheter towards the proximal direction.

The catheter can further be configured such that the proximal rolling membrane can at least in part fold onto itself such that overlapping portions of the membrane can be formed.

In some cases, the catheter can be adapted such that overlapping portions are at least formed when the inner shaft is moved in a distal direction relative to the outer shaft. Alternatively, it can also be possible that overlapping portions are formed intrinsically independent of a relative motion of the inner shaft to the outer shaft.

In the latter case, it can be possible that the proximal end of the proximal rolling membrane is initially folded inwards (the proximal end of the membrane can thus initially be folded onto itself). A distal-most portion of the inwards folded portion of the proximal rolling membrane can then be folded outwards. Therefore, a portion of the proximal rolling membrane can be formed at its proximal end at which the respective folded portions of the proximal rolling membrane overlap twice.

By folding the proximal rolling membrane onto itself and such that overlapping portions of the proximal rolling membrane are formed in an attachment portion (in which the proximal rolling membrane can be attached to the inner shaft), an overlap of the proximal rolling membrane can be maintained, even under a repeated relative movement of the inner shaft relative to the outer shaft such that a blocking of the proximal rolling membrane can be avoided. This can advantageously contribute to a friction-less movement of the inner shaft relative to the outer shaft.

The catheter can be adapted such that overlapping portions of the proximal rolling membrane are at least in part attached to each other by an adhesive. In some cases, the adhesive can be a glue.

Filling the overlap with an adhesive can advantageously contribute to ensuring that the overlap is maintained even after multiple movements (e.g., in a distal direction and/or a proximal direction) of the inner shaft relative to the outer shaft.

In some cases, the proximal rolling membrane can be adapted as a first rolling membrane for rolling along the outer shaft and/or a blood vessel of a patient.

The rolling membrane is a membrane which is movable along an axial direction. An axial direction can be understood as a direction along a longitudinal extension (e.g. defined by a direction from the proximal end to the distal end of the catheter) of the catheter (and thus of the inner shaft and the outer shaft). The rolling membrane can be understood as a membrane which can be implemented similar to a hose wherein at least a portion of the rolling membrane can be folded inwards into itself in a retracted state and wherein at least a portion of the inwards folded portion of the rolling membrane can be folded outwards (and thus rolled out) as a result of applying a force onto an inner wall of the rolling membrane in a direction along which the rolling membrane is intended to be rolled out (e.g., the proximal direction). A force, which can lead to a rolling out of the proximal rolling membrane (exemplarily adapted as a rolling membrane), can be applied by moving the inner shaft in a proximal direction relative to the outer shaft. Additionally or alternatively, a hydrostatic pressure acting on an inner wall of the rolling membrane into, e.g., the proximal direction, can at least contribute to or initiate a rolling out of the rolling membrane in the proximal direction. In analogy to the aforementioned, a retraction of the rolling membrane can be understood as a movement of the inner shaft relative to the outer shaft in the proximal direction.

By adapting the proximal rolling membrane as a rolling membrane the proximal rolling membrane can be rolled out along a blood vessel of a patent and/or outside the body a patient (and/or within the outer shaft, preferably in the proximal direction). Therefore, a rolling out of the rolling membrane can be simplified.

The catheter can be adapted such that the proximal rolling membrane cannot leave the outer shaft, preferably in a distal direction. The proximal rolling membrane can have a length which is shorter than a length of the outer shaft. The proximal rolling membrane and/or the distal rolling membrane can have a length of between 150 mm and 100 mm, preferably 120 mm. The proximal rolling membrane and the distal rolling membrane can have the same length.

In some cases, the catheter can be adapted such that the proximal rolling membrane can remain in the outer shaft at any time, e.g., the proximal rolling membrane can remain in the outer shaft if the inner shaft is fully inserted into to the outer shaft (e.g., the inner shaft is moved to a distal-most position relative to the outer shaft) and the proximal rolling membrane can remain in the outer shaft if the inner shaft is fully pulled out (e.g., the inner shaft can be moved to a proximal-most position) relative to the outer shaft.

Such an arrangement can ensure a protection of the proximal rolling membrane within the outer shaft (e.g., during an insertion or retraction procedure of the catheter into/out of the blood vessel of the patient) to avoid any damages to the proximal rolling membrane which can disadvantageously affect its properties (e.g., which can cause leakage). Moreover, by adapting the catheter such that the proximal rolling membrane cannot leave the outer shaft, a well-defined environment can be defined for the proximal rolling membrane in which it can expand under controlled conditions (e.g., the proximal rolling membrane can thus not be blocked by a local stenosis of a blood vessel and/or any other obstacles). Another advantage of such a catheter the proximal rolling membrane, e.g. the rolling membrane, is in contact with the outer shaft under pressure and the static friction prevents accordion-like folding instead of a rolling.

The catheter can further include a second membrane, preferably adapted as a rolling membrane, at a distal end of the catheter. Therefore, the second membrane is also referred to as a distal rolling membrane.

The distal rolling membrane can be attached to the inner shaft and to the outer shaft. The distal rolling membrane can be attached to the distal inner shaft portion. The distal rolling membrane can be attached to the distal outer shaft portion. The distal rolling membrane can be attached to a distal end of the inner shaft and can be attached to a distal end of the outer shaft.

The distal rolling membrane can be a second rolling membrane which is spatially separated from the proximal rolling membrane. Alternatively, the distal rolling membrane is connected to the proximal rolling membrane or the distal rolling membrane and the proximal rolling membrane are integrally formed.

In some embodiments, the proximal rolling membrane can be attached to a first outer shaft, and the distal rolling membrane can be attached to a second outer shaft. The first outer shaft can be arranged proximally to the second outer shaft. The first outer shaft and the second outer shaft can be attached to a first handle of the catheter and the inner shaft can be connected to a second handle. The first handle and/or the second handle can include a port (for injection of a fluid and or for inflation or deflation of the rolling membrane).

The distal rolling membrane can generally be adapted to include aspects described herein with respect to the proximal rolling membrane (e.g., with respect to the material, rolling out characteristics, etc.).

The catheter can be adapted such that a portion of the proximal rolling membrane attached to the inner shaft can be moved distally, when the distal rolling membrane is rolled out distally.

In some cases, the proximal rolling membrane can be adapted to move in a functionally opposing manner as compared to the distal rolling membrane. Said effect can be based on the position of the attachment of the proximal rolling membrane to the inner shaft and of the distal rolling membrane to the inner shaft accordingly on the respective proximal and distal ends of the catheter, such that, e.g., the distal rolling membrane can be in a fully retracted state when the proximal rolling membrane is in a fully rolled out state.

In some cases, a single lumen can be defined by the respective inner walls of the proximal rolling membrane and the distal rolling membrane, wherein, in some cases, the lumen can be filled with a fluid. Such a fluid filling can allow a communication between the proximal rolling membrane and the distal rolling membrane such that any movement of the proximal rolling membrane can be translated into an opposing movement of the distal rolling membrane.

Such an arrangement can allow for a communication of the proximal rolling membrane and the distal rolling membrane such that the distal rolling membrane (which cannot be accessed during, e.g., a coronary intervention) can essentially be remote-controllable by the proximal rolling membrane (which can, in some cases, be located outside of the body of the patient). Therefore, an improved usability of the catheter can be provided.

A diameter of the proximal rolling membrane can be smaller than a diameter of the distal rolling membrane. The diameter of the proximal rolling membrane can be less than 2.0 mm, preferably less than 1.67 mm.

The diameter of the proximal rolling membrane and the distal rolling membrane can be understood as twice the (shortest) radially measured distances between a center axis of the proximal rolling membrane or the distal rolling membrane and an outer wall of the proximal rolling membrane or the distal rolling membrane, respectively.

By providing the proximal rolling membrane with a diameter which is smaller than a diameter of the distal rolling membrane, an efficient force transfer between forces acting on the proximal rolling membrane from the proximal direction towards the distal direction onto the distal rolling membrane (in a distal direction) can be facilitated. Moreover, a net-amplification of a force acting on the distal rolling membrane can be facilitated as it will be described further below.

The proximal rolling membrane and/or the distal rolling membrane can be liquid tight. Liquid tight can be understood as being impermeable for liquids (e.g., water, rinsing fluids, heparinized fluids, blood, etc.). The term liquid tight can not be limited to the liquid tight properties of the proximal rolling membrane and/or the distal rolling membrane. In some cases, also the attachment of the proximal rolling membrane to the inner shaft and the outer shaft can be configured to be liquid tight.

In some cases, the proximal rolling membrane and/or the distal rolling membrane (and their respective attachment points on the inner shaft and on the outer shaft) can also be fluid tight (e.g., impermeable against liquids and/or gases (e.g., air)).

By adapting the proximal rolling membrane and/or the distal rolling membrane such that it is liquid tight (or fluid tight), a sealing of the catheter at a respective portion (distal/intermediate/proximal) of the catheter at which the proximal rolling membrane and/or the distal rolling membrane is situated can be ensured such that a liquid (or fluid) cannot flow past the proximal rolling membrane and/or the distal rolling membrane. Such an arrangement can in particular render the usage of a haemostatic valve at a proximal end of the catheter obsolete, which further decreased the complexity of operating the catheter.

The proximal rolling membrane and/or the distal rolling membrane can be adapted to be rolled-out and/or retracted in an essentially friction-less manner. In some examples, the distal rolling membrane is attached to an outer side of an outer shaft, whereas the proximal rolling membrane can be attached to an inner side of an outer shaft (not necessarily the same outer shaft to which the distal rolling membrane is attached).

As a result of moving the inner shaft in a distal direction relative to the outer shaft, overlapping portions of the distal rolling membrane can be formed at a proximal end of the catheter (proximal rolling membrane) whereas overlapping portions of the distal rolling membrane can be formed at a distal end of the catheter if the inner shaft is moved in a proximal direction relative to the outer shaft.

In some cases, the proximal rolling membrane and/or the distal rolling membrane can be made from a material which has a low coefficient of friction when portions of the respective material are scrubbed relative to each other. Alternatively or additionally, the surface of the portions of the proximal rolling membrane and/or the distal rolling membrane which undergo an overlap can be provided with a friction reduction coating (e.g., by a hydrophilic coating).

Additionally or alternatively, the overlapping portions can be exposed to a fluid (e.g., water and/or any other suitable fluid) to reduce the coefficient of friction of the respective portions if they are scrubbed relative to each other as a potential result of a relative movement of the inner shaft and the outer shaft.

By providing the proximal rolling membrane and/or the distal rolling membrane such that a friction-less rolling out and/or retraction of the membrane is ensured, an easy to move movability of the inner shaft and the outer shaft can be facilitated.

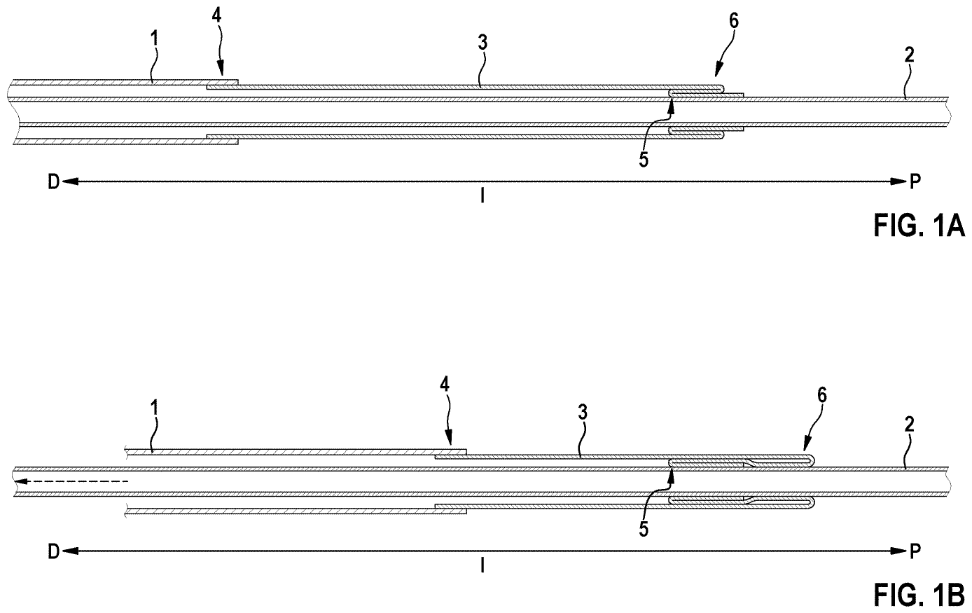

FIGS. 1A and 1B show an exemplary embodiment of a catheter in a cross-sectional view, taken along a longitudinal direction of the catheter.

More specifically, FIG. 1A shows the catheter according to a first embodiment in which it can include an outer shaft 1 and an inner shaft 2, wherein the inner shaft 2 can be situated at least in part in the outer shaft 1. The outer shaft 1 and the inner shaft 2 can be connected with each other by a proximal rolling membrane 3. The situation depicted in FIG. 1A relates to a situation in which the inner shaft 2 is pulled out from outer shaft 1 to a proximal position which can be a proximal-most position.

At least one of the outer shaft 1 and/or the inner shaft 2 can be provided with a circular cross-section (as it can be obtained from a cut substantially transverse to a longitudinal direction of the catheter). The catheter, the outer shaft 1, the inner shaft 2 and the proximal rolling membrane 3 can include a distal end D, which can be located closer to a distal end D of the catheter (and/or the outer shaft 1 and/or the inner shaft 2 and/or the proximal rolling membrane 3) as compared to a proximal end P which can be located on an opposing end of the catheter (and/or the outer shaft 1 and/or the inner shaft 2 and/or the proximal rolling membrane 3 relative to the longitudinal extension of the catheter and/or the outer shaft 1 and/or the inner shaft 2 and/or the proximal rolling membrane 3).

The distal end of the proximal rolling membrane 3 can be attached to the outer shaft 1 at a first attachment point 4. The proximal end of the proximal rolling membrane 3 can be attached to the inner shaft 2 at a second attachment point 5. In some cases, the proximal end P of the proximal rolling membrane 3 can directly be attached to the inner shaft 2. As depicted in FIG. 1A, the proximal end P of the proximal rolling membrane 3 can at least partially be folded inwards, wherein a portion of the inwards folded portion can then be folded outwards again such that the proximal end of the proximal rolling membrane 3 is essentially directed to the proximal direction of the catheter when attached to the inner shaft 2 at the second attachment point 5. As a result of the aforementioned folding of the proximal rolling membrane 3 an overlapping portion 6 of the rolling membrane 3 is formed. Notably, two overlapping layers are formed, situated above one another (e.g., one overlap can be closer to an outer surface of the inner shaft wherein the respective second overlap is situated further apart from the outer side of the inner shaft 2).

The first attachment point 4 can be situated at a proximal-most portion or end of the outer shaft 1. However, in some cases, it can also be possible that the proximal rolling membrane 3 is attached to the outer shaft at a more distal location of the outer shaft 1 (e.g., distanced from the proximal-most end of the outer shaft 1 by 1 cm, 2 cm, 3 cm, etc., in a more distal direction). Preferably, the first attachment point 4 can be situated inside the outer shaft 1. Alternatively, it can also be possible that the first attachment point 4 is located on an outer side of the outer shaft 1.

The second attachment point 5 can preferably be located at an intermediate inner shaft portion I of the inner shaft 2. The intermediate inner shaft portion I can be situated in between the distal inner shaft portion D of the inner shaft 2 and the proximal inner shaft portion P of the inner shaft 2. The second attachment point 5 can preferably be located on the outer side of the inner shaft 2.

FIG. 1B shows the catheter of FIG. 1A in a situation in which the inner shaft 2 has been moved into the outer shaft 1 along the distal direction D. As a result of moving the inner shaft 2 further into the outer shaft 1 in a distal direction D, the overlapping portion 6 of the proximal rolling membrane 3 (at the second attachment point 5) is increased.

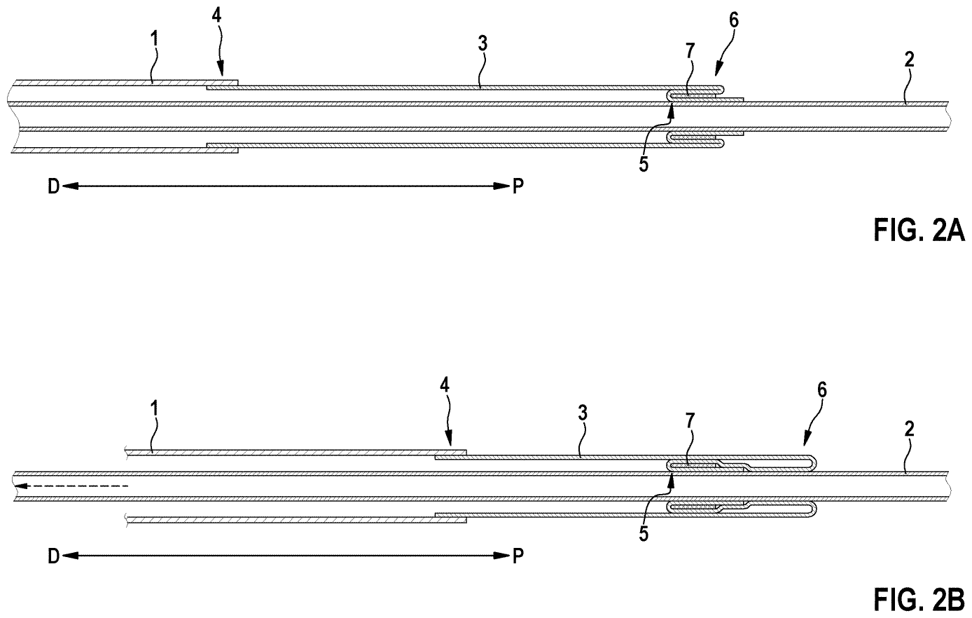

FIGS. 2A and 2B show another exemplary embodiment of a catheter, wherein the catheter can be implemented identically to the catheter as described with reference to FIGS. 1A and 1B, above. It is noted that the catheter according to the embodiment described with reference to FIGS. 2A and 2B can not be limited to the aspects of this embodiment but can also be combined with aspects of other embodiments as described herein.

In analogy to FIG. 1A, FIG. 2A shows the catheter in a situation in which the inner shaft 2 has been moved to a proximal position which can be a proximal-most position. FIG. 2A exemplarily depicts the proximal rolling membrane 3 in a (fully) rolled-out state. However, in the embodiment shown in FIG. 2A, an overlap of the overlapping portion 6 of the proximal rolling membrane 3 (at the second attachment point 5) can be filled (at least in part) with an adhesive 7 such as to permanently maintain the overlap of the proximal rolling membrane 3 at the second attachment point 5. Preferably, the overlap which is closer to the outer side of the inner shaft 2 can be filled with the adhesive 7.

In any case, the proximal rolling membrane 3 can be attached to the outer shaft 1 (at attachment point 4) and/or to the inner shaft 2 (at attachment point 5) by one or more of welding, gluing, screwing, shrinking and/or any other suitable attachment method. Alternatively, in some cases, the proximal rolling membrane 3 can be attached to the outer shaft 1 by blow molding the proximal rolling membrane 3 from the outer shaft 1.

FIG. 2B (in analogy to FIG. 1B) depicts the catheter of FIG. 2A, wherein the inner shaft 2 has been moved relative to the outer shaft 1 in a distal direction. As a result of the movement of the inner shaft 2 relative to the outer shaft 1 in the distal direction D, the overlapping portion 6 can be increased (with respect to metric length units). Due to the permanent maintaining of the overlap of the overlapping portion 6 which is situated closer to the outer side of the inner shaft 2, the inwards folded portion 6 can be maintained during a relative movement of the inner shaft 2 relative to the outer shaft 1. In the situation, depicted in FIG. 2B, the proximal rolling membrane 3 can be understood as being in a partially retracted state.

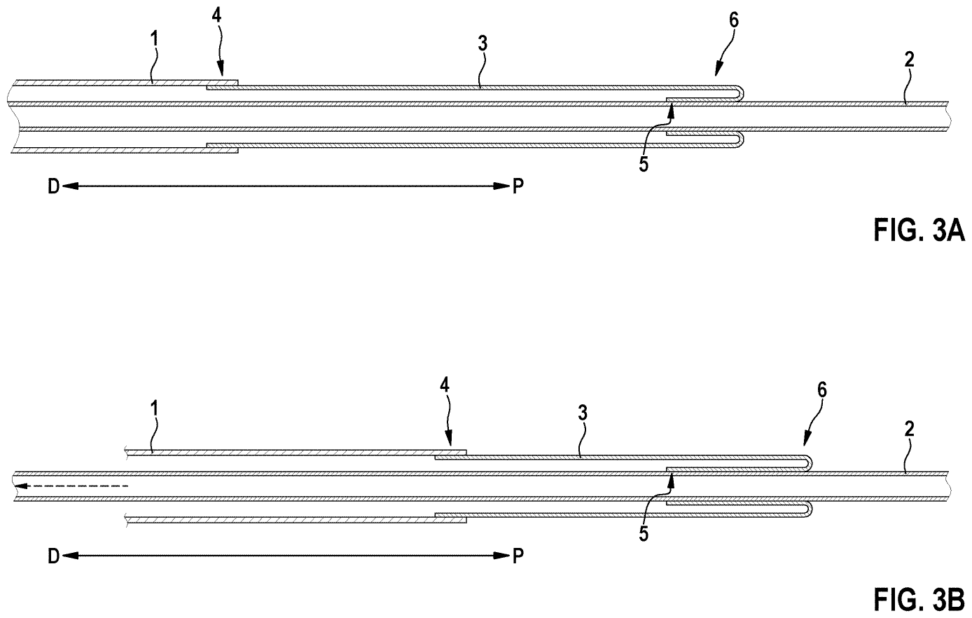

FIGS. 3A and 3B show another exemplary embodiment of the catheter wherein the catheter can be implemented as described with reference to FIGS. 1 and 2, above. It is noted that the catheter according to the embodiment described with reference to FIGS. 3A and 3B can not be limited to the aspects of this embodiment but can also be combined with aspects of other embodiments described herein.

In analogy to FIG. 1A and FIG. 2A, FIG. 3A shows the catheter in a situation in which the inner shaft 2 has been moved to a proximal(-most) position. In particular, FIG. 3A depicts an embodiment of the catheter in which the proximal rolling membrane 3 is attached to the inner shaft 2 such that the proximal-most end of the proximal rolling membrane 3 is directed towards the distal direction. Hence, the membrane is generally folded inwards (at its respective proximal end).

In a preferred implementation, only a single overlap can be formed at the overlapping portion 6. The shape of overlapping portion (as seen in a cross-sectional view of the catheter taken along a longitudinal axis) can be understood as an axially mirrored letter “C”.

FIG. 3B (in analogy to FIGS. 1B and 2B) shows the catheter of FIG. 3A wherein the inner shaft 2 has been moved relative to the outer shaft 1 in the distal direction D such that the overlapping portion 6 can be increased.

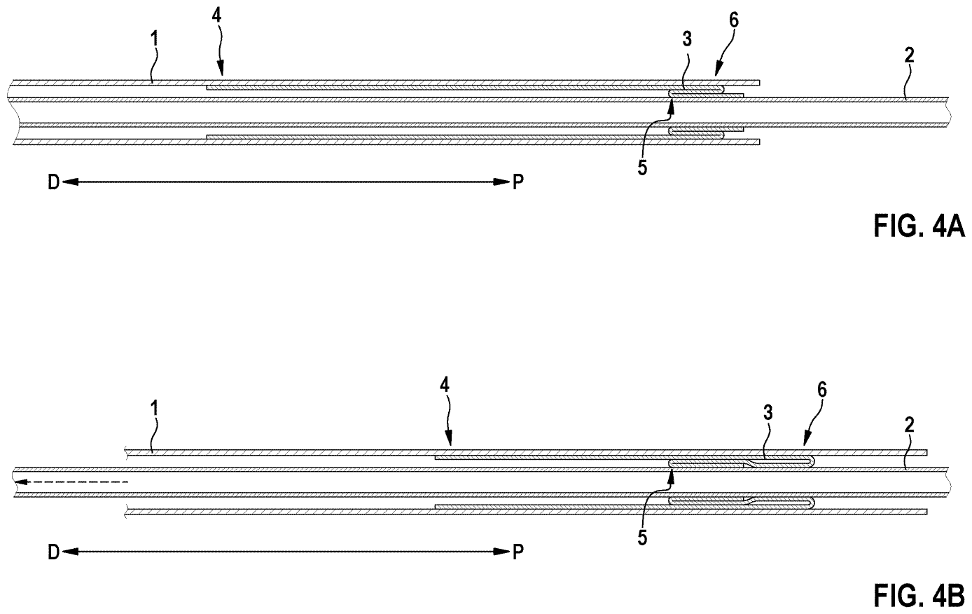

FIGS. 4A and 4B show another exemplary embodiment of the catheter, wherein the catheter can be implemented as described with reference to FIGS. 1-3, above. It is noted that the catheter according to the embodiment described with reference to FIGS. 3A and 3B can not be limited to the aspects of this embodiment but can also be combined with aspects of other embodiments described herein.

In analogy to FIG. 1A, FIG. 2A and FIG. 3A, FIG. 4A shows the catheter in a situation in which the inner shaft 2 has been moved to a proximal(-most) position.

FIG. 4A shows an exemplary implementation in which the catheter is configured such that the first attachment point 4 and the second attachment point 5 are situated such that the proximal rolling membrane 3 can remain within the outer shaft 1, e.g. at all times, e.g., also when the inner shaft 2 is moved to a proximal-most position. The aforementioned aspect can be achieved by configuring the catheter such that the first attachment point 4 can be situated at a location which is situated further in the distal direction D (inside the outer shaft 1) as compared to the location of the first attachment point 4 as described with respect to the aforementioned embodiments of the catheter (FIGS. 1-3, above). In other words, the location of the first attachment point 4 can be chosen such that overlapping portion 6 can still be inside the outer shaft 1 when the inner shaft 2 has been moved to a proximal-most position (and if the proximal rolling membrane 3 has thus been rolled out). Such an arrangement can in particular ensure that the inwards folded portion of the proximal rolling membrane 3 is maintained even under pressure.

It is further noted that the first attachment point 4 and/or the second attachment point 5 can be attached to the outer shaft 1 and/or the inner shaft 2, respectively, at a single point or, alternatively, along a spatially extending attachment area (e.g., the first attachment point 4 and/or the second attachment point 5 can be attached to the outer shaft 1 and/or the inner shaft 2 along a portion of the proximal rolling membrane 3 which has a length of 1 cm, 2 cm, 3 cm or any other suitable length).

In some cases, the outer shaft 1 can be a single shaft onto which the proximal rolling membrane 3 can be attached. Alternatively, it can also be possible that the outer shaft 1 is made from at least two distinct elements such that at least one of the at least two distinct elements is situated at a further distal portion of the catheter whereas the at least one other respective element is situated at a further proximal portion of the catheter. Such an arrangement can, e.g., simplify the manufacturing of the catheter as the distally located element of the outer shaft 1 can be manufactured as a tubular element whereas the proximally located element can be adapted such that the proximal rolling membrane 3 is, e.g., mold blown from the proximally element of the outer shaft 1. Therefore, the manufacturing efforts can essentially be limited to the proximally located element only (e.g., with respect to the handling of the distally located portion which can have to be inserted into a respective mold blowing apparatus).

FIG. 4B (in analogy to FIGS. 1B, 2B and 3B) shows the catheter of FIG. 4A wherein the inner shaft 2 has been moved relative to the outer shaft 1 in the distal direction D such that the overlapping portion 6 can be increased.

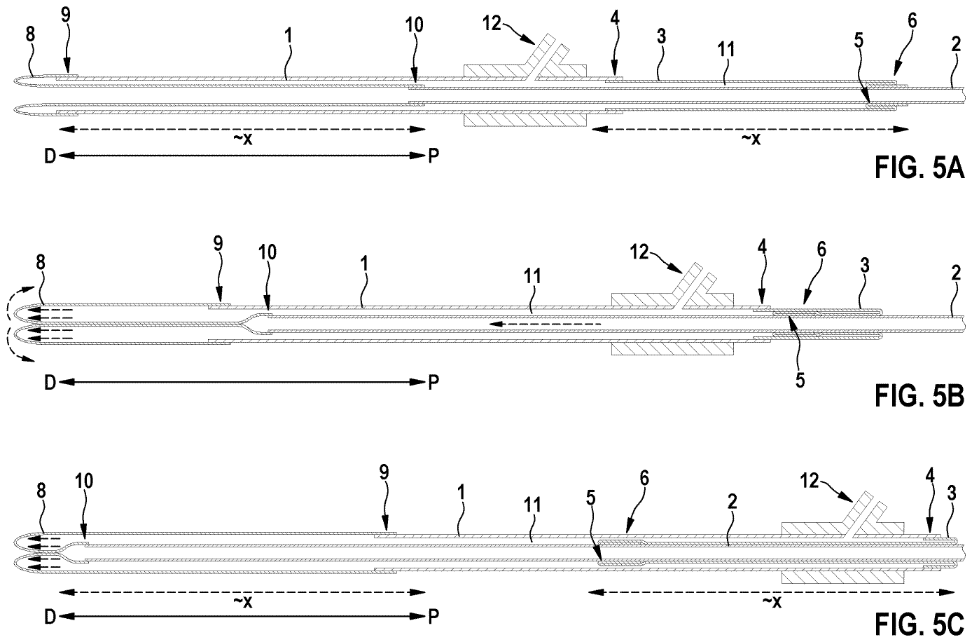

FIGS. 5A-5C show another exemplary embodiment of the catheter in which the catheter can be provided with a proximal rolling membrane 3 on the proximal end of the outer shaft 1, identical to one of the embodiments as described with reference to any of FIGS. 1-4, above. It is noted that the catheter according to the embodiment described with reference to FIGS. 5A and 5B can not be limited to the aspects of this embodiment but can also be combined with aspects of other embodiments described herein.

In analogy to FIG. 1A, FIG. 2A, FIG. 3A and FIG. 4A, FIG. 5A shows the catheter in a situation in which the inner shaft 2 has been moved to a proximal(-most) position.

Besides providing the catheter with the proximal rolling membrane 3, in addition, the catheter can optionally also be provided with a distal rolling membrane 8 (preferably implemented as a rolling membrane) on a distal end of the catheter. At least a distal most portion of the distal rolling membrane 8 can be folded into itself (in a retracted state).

The distal rolling membrane 8 can be attached to the outer shaft at a distal-most portion of the outer shaft 1 at a third attachment point 9. The third attachment point 9 can preferably be located on the outer side of the outer shaft 1. Alternatively, in some cases, the third attachment point 9 can also be located inside the outer shaft 1.

The distal rolling membrane 8 can further also be attached to a distal-most portion of the inner shaft 2 at a fourth attachment point 10 by means of the inwards folded portion of the distal rolling membrane 8. The fourth attachment point 10 can preferably be located on an outer side of the inner shaft 2.

FIG. 5B (in analogy to FIGS. 1B, 2B, 3B and 4B) shows the catheter of FIG. 5A wherein the inner shaft 2 has been moved relative to the outer shaft 1 in the distal direction D such that the overlapping portion 6 can be increased.

A lumen 11 can further be defined between a distal end of the distal rolling membrane 8 and a proximal end of the proximal rolling membrane 3. The lumen 11 can be sealed by a respective inner wall of the distal rolling membrane 8 and the proximal rolling membrane 3. As a result of moving the inner shaft 2 relative to the outer shaft 1, the distal-most end of the inner shaft 2 (and thus the respective fourth attachment point 10) can also be moved in a distal direction D, thus contributing to a rolling out of the distal rolling membrane 8 along the distal direction D.

Moreover, since the lumen 11 can be provided with a constant pressure (e.g., by means of a valve 12), a movement of the proximal-most portion of the proximal rolling membrane 3 can apply a certain force onto the lumen 11 (effectively acting in a distal direction) and thus to the distal-most portion of the distal rolling membrane 8 which can thus contribute (in addition or let alone) to a rolling out of the distal rolling membrane 8. Thus, if the first (proximal) rolling membrane 3 is (significantly) smaller in diameter than the second (distal) rolling membrane, the presence of hydraulic pressure in the lumen 11 is already sufficient to exert a net force on the inner shaft 2 in the distal direction. This will ‘automatically’ move the inner shaft 2 in the distal direction relative to the outer shaft 1. Therefore, it can be considered as a hydraulic drive. Valve 12 can further be used to provide the lumen 11 with at least one fluid (e.g., liquids (water, heparinized liquids, etc.) and/or at least one gas (e.g., air). Valve 12 can preferably be situated on the outer shaft 1 and can preferably be located outside of the body of the patient. Therefore, also at least a proximal outer shaft portion of the outer shaft 1 can be located outside of the body of the patient.

Independently of whether a distal rolling membrane 8 is present, the catheter according to FIGS. 5A-C can include a handle configured to be arranged outside the patient, wherein valve 12 can be part of the handle. The distal rolling membrane 3 can be arranged within the handle, e.g. outside the patient. Via the valve an inflation or deflation of the distal rolling membrane can be achieved.

In some examples, the proximal rolling membrane 3 can be attached to a first outer shaft, and the distal rolling membrane 8 can be attached to a second outer shaft. The first outer shaft can be arranged proximally to the second outer shaft. Both outer shafts can be attached to a handle of the catheter, for example.

FIG. 5C shows the catheter of FIGS. 5A and 5B, wherein the inner shaft 2 has been moved to a distal-most position. In said configuration, the second attachment-point 5 has been moved to a distal-most location as well.

As depicted in FIGS. 5A-5C, the catheter can be adapted such that the proximal rolling membrane 3 and the distal rolling membrane 8 can move opposed to each other. In other words, when the proximal rolling membrane 3 is in a fully rolled-out state (FIG. 5A, inner shaft 2 has been moved to a proximal-most position), the distal rolling membrane 8 is in a fully retracted state. In contrast, when the proximal rolling membrane 3 is in a full retracted state (FIG. 5C, inner shaft 2 has been moved to a proximal-most position), the distal rolling membrane 8 can be in a rolled-out state.

In a preferred implementation, the distal rolling membrane 8 can be provided with the same length as the proximal rolling membrane 3, as measurable in a fully rolled-out state of each of the two membranes. This allows for a fully opposing movement of the proximal rolling membrane 3 relative to the distal rolling membrane 8 (i.e., the distal rolling membrane 8 can be fully rolled out when the proximal rolling membrane is fully retracted and vice versa).

In some cases, the diameter of the proximal rolling membrane 3 can be smaller than the diameter of the distal rolling membrane 8. Such a configuration can allow the distal movement of the inner shaft 2 via a hydraulic pressure. Notably, a force F_{fm} can be applied to the proximal rolling membrane 3 (e.g., by applying the respective force onto the inner shaft and/or an outer wall of the proximal rolling membrane in a distal direction) which can be provided with a certain radius which can be associated with a cross sectional area A_{fm} of the proximal rolling membrane. Said force can be transferred to the distal rolling membrane 8 with a cross sectional area Amm, wherein A_{fm}<Aᵣₘ. Under the assumption of a constant pressure in the lumen 11 defined by the proximal rolling membrane 3 and the distal rolling membrane 8, the respective force Fᵣₘ acting on an inner wall of the distal rolling membrane 8 can depend on F_{fm} since:

$$\frac{{F}_{fm}}{{A}_{fm}} = \frac{{F}_{rm}}{{A}_{rm}}$$

Therefore, it can be derived that

$${F}_{rm} = {F}_{fm} \cdot \frac{{A}_{rm}}{{A}_{fm}}$$

Since A_{fm}<Aᵣₘ, a net-increase of the force Fᵣₘ relative to F_{fm} can be obtained which can contribute to a simplified handling of the catheter. This enables a simplified handling or unrolling of the second rolling membrane. In addition, a force can be realized at the distal end of the catheter (and thus in the patient's body) by means of the second rolling membrane, which is for example usable to cross a stenosis.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A catheter, comprising:
an inner shaft and an outer shaft, wherein the inner shaft comprises a distal inner shaft portion and a proximal inner shaft portion arranged proximally to the distal inner shaft portion; and
a proximal rolling membrane attached to the proximal inner shaft portion and to the outer shaft.

2. The catheter according to claim 1, wherein the outer shaft comprises a distal outer shaft portion and a proximal outer shaft portion arranged proximally to the distal outer shaft portion; and wherein the proximal rolling membrane is attached to the proximal outer shaft portion.

3. The catheter according to claim 1, wherein the inner shaft and the outer shaft are configured to be movable relative to each other along an axial direction.

4. The catheter according to claim 1, wherein the proximal rolling membrane comprises a length configured such that the proximal rolling membrane cannot leave the catheter in a distal direction.

5. The catheter according to claim 1, wherein the proximal rolling membrane comprises a portion for an attachment to the inner shaft, wherein an end of the portion faces a distal direction.

6. The catheter according to claim 1, wherein the proximal rolling membrane comprises a portion for attachment to the inner shaft, wherein an end of the portion faces a proximal direction.

7. The catheter according to claim 1, wherein the proximal rolling membrane can at least in part fold onto itself such that at least one overlapping portion of the proximal rolling membrane is formed.

8. The catheter according to claim 7, wherein the at least one overlapping portion of the proximal rolling membrane comprises at least two portions at least in part attached to each other by an adhesive.

9. The catheter according to claim 1, wherein the proximal rolling membrane is configured along the outer shaft and/or a blood vessel of a patient.

10. The catheter according to claim 1, wherein the proximal rolling membrane has a length which is shorter than a length of the outer shaft.

11. The catheter according to claim 1, wherein the catheter comprises a distal rolling membrane at a distal end of the catheter.

12. The catheter according to claim 11, wherein the distal rolling membrane is attached to the inner shaft and to the outer shaft or a second outer shaft.

13. The catheter according to claim 11, wherein the distal rolling membrane is attached to the distal inner shaft portion.

14-20. (canceled)

21. The catheter according to claim 12, wherein the proximal rolling membrane and/or the distal rolling membrane are adapted configured to be rolled out in the axial direction.

22. A catheter, consisting of:
an inner shaft and an outer shaft, wherein the inner shaft consists of a distal inner shaft portion and a proximal inner shaft portion arranged proximally to the distal inner shaft portion;
one or both of a proximal rolling membrane and a distal rolling membrane, wherein the proximal rolling membrane is attached to the proximal inner shaft portion and to the outer shaft and the distal rolling membrane is attached to the distal inner shaft portion and to the outer shaft or a second outer shaft.

23. The catheter according to claim 22, wherein the proximal rolling membrane can at least in part fold onto itself such that at least one overlapping portion of the proximal rolling membrane is formed.

24. The catheter according to claim 22, wherein the at least one overlapping portion of the proximal rolling membrane comprises at least two portions at least in part attached to each other by an adhesive.
